(19) United States
(12) Patent Application Publication
(10) Pub. No.: US 2016/0317662 A1
(43) Pub. Date: Nov. 3, 2016

(54) STABLE ORAL PHARMACEUTICAL COMPOSITION

(71) Applicant: HYPERMARCAS S.A., São Paulo (BR)

(72) Inventors: Walker Magalhães LAHMANN, São Paulo (BR); Hilton Oliveira DOS SANTOS FILHO, São Paulo (BR); Iara Silvia Brauer MANTOVANI, Anápolis City (BR); Cristiano Martins VELOSO, Anápolis (BR); Ricardo Alves DE OLIVEIRA, Anápolis (BR); Amanda Junqueira MANCILHA, Anápolis (BR)

(21) Appl. No.: 15/105,299

(22) PCT Filed: Dec. 16, 2014
(86) PCT No.: PCT/BR2014/050046

§ 371 Date: Jun. 16, 2016

(30) Foreign Application Priority Data
Dec. 16, 2013 (BR) 1020130323950
Feb. 17, 2014 (BR) BR1020140036865

(51) Int. Cl. A61K 47/10; A61K 31/4152; A61K 47/12; A61K 9/28; A61K 47/02; A61K 9/00; A61K 31/485

(52) U.S. Cl. CPC A61K 47/10; A61K 9/0053; A61K 31/4152; A61K 31/485; A61K 9/2853; A61K 47/02; A61K 47/12

(57) ABSTRACT

The present invention relates to a stable oral pharmaceutical composition for relieving moderate to intense pain, related to trauma (sprains, dislocations, contusions, distensions, fractures), postoperative and post-tooth extraction conditions, neuralgia, lumbago, joint pain and similar conditions. Such composition comprises (a) dipyrone, its free acid or pharmacologically acceptable salts thereof, (b) codeine (7,8-dihydro-4,5-epoxy-3-methoxy-17-methyl morfinane-6-ol) or pharmacologically acceptable salts, (c) polyethylene glycol and (d) pharmaceutically acceptable excipients, in small amounts. Particularly, the present invention relates to a coated tablet.

### FIELD OF THE INVENTION

The present invention relates to a stable oral pharmaceutical composition for relieving moderate to intense pain, related to trauma (sprains, dislocations, contusions, distensions, and fractures), postoperative and post-tooth extraction conditions, neuralgia, lumbago, joint pain and similar conditions. Said composition comprises (a) dipyrone, its free acid or pharmacologically acceptable salts thereof, (b) codeine or pharmacologically acceptable salts, (c) polyethylene glycol and (d) pharmaceutically acceptable excipients, in small amounts. Particularly, the present invention relates to a coated tablet.

### BACKGROUND OF THE INVENTION

Although there are many pharmaceutical compositions focused on analgesia of painful conditions, most of them still employs a single active principle, due to technical difficulties in the preparation of compositions comprising associations. In many cases, in order to provide analgesic efficacy, especially in cases of moderate to intense pain, it is necessary to deliver two or more pharmaceutical compositions to the patient, in order to provide two or more active principles.

There is a constant search for associations of active principles able to provide a safe and effective analgesic effect, particularly to the relief of pain arising from conditions associated with trauma (sprains, dislocations, contusions, distensions, and fractures), postoperative and post-tooth extraction conditions, neuralgia, lumbago, joint pain and similar conditions.

The state of the art indicates, in the case of lack of response to non-narcotic anti-inflammatory analgesics, its combination with weak opioids.

### Non-Narcotic Anti-Inflammatory Analgesics

Phenyl pyrazolones are among the most used analgesics. Dipyrone sodium monohydrate (formula I) is a non-narcotic pyrazolone derivative that has analgesic and antipyretic effects. It is a prodrug, which needs to be hydrolyzed in the gastrointestinal tract to form the active metabolite (4-N-methylaminoantipyrine).

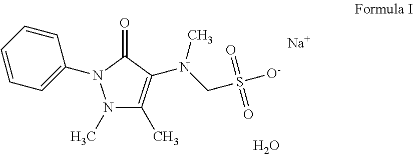 [SMILES: Cc1c(N(C)CS(=O)(=O)[O-])c(=O)n(-c2ccccc2)n1C ; [Na+] ; O]

Dipyrone is also a notoriously unstable molecule in aqueous medium. Its main degradation products, by hydrolysis, are the n-methyl-aminophenazone or 4-N-methylaminoantipyrine (4-MAA), sodium metabisulfite and formaldehyde which, in turn, are very reactive products that present potential for reaction with various molecules.

The stability of dipyrone may be mainly affected by the temperature and pH of the medium. The rate of hydrolysis of the molecule increases dramatically under acid solutions, compared to more alkaline solutions. The instability of dipyrone is evidenced since its formulations becomes yellow colored.

Opioid Analgesics

Opium-derived analgesics are commonly used to relieve moderate to severe pain. Opiates cause analgesia by binding to receptors of encephalin or endorphins in the brain, spinal cord and peripheral nervous system. They act by the modulation of nociceptive activity through various mechanisms. They comprise a heterogeneous group of natural, synthetic and endogenous drugs (encephalin, endorphins and dinorphins) with chemical properties similar to morphine. They are classified as agonists (morphine, codeine, heroin, oxycodone, methadone, propoxyphene, meperidine, phentanyl), partial agonists (buprenorphine), agonist/antagonist (pentazocine, nalbuphine) and antagonist (naloxone), according to their ability to sensibilize opiate receptors.

The codein phosphate hemihydrate (formula II) is a pro-drug whose analgesic effect depends on hepatic metabolism, with the formation of morphine as an active metabolite. Notoriously unstable, it degrades to form dimers, in an oxidative process. Its salts are highly soluble in water and the aqueous solutions are acidic.

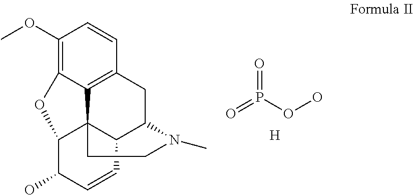

Codeine phosphate solutions are relatively stable around pH 3.5. The state of the art teaches that a gradual increase in the degradation rate of codeine phosphate solutions occur, with pH increase from 1.6 to 10.5.

In general, the provision of two or more active principles in a single pharmaceutical form (fixed dose associations) provides greater convenience to the patient, insofar as it allows the ingestion of just one tablet, instead of two. Moreover, this reduces the cost of industrial production and regulatory processes.

However, in a usual manner, its production presents technical difficulties especially related to: different solubilities of the active principles; different dissolution profiles; incompatibilities among the active principles and of these with the excipients; different stability conditions, among others. In view of the difficulties verified, a common problem is to maintain the performance that active principles have individually, when they are combined in the same pharmaceutical form, comprising two active principles and excipients.

Thus, although literature points out the enhanced effect of non-narcotic anti-inflammatory analgesics combined with opiate analgesics, there is still the need of providing a stable oral pharmaceutical composition comprising a combination of the same.

### BRIEF DESCRIPTION OF THE INVENTION

The present invention relates to a stable oral pharmaceutical composition comprising a) dipyrone, its free acid or its pharmacologically acceptable salts; b) codeine or its pharmacologically acceptable salts; c) polyethylene glycol; d) pharmaceutical excipients in small amounts, in which said composition has pH of about 6.5.

### BRIEF DESCRIPTION OF THE FIGURES

FIG. 1 presents the dissolution profile of tablets comprising 1000 mg of dipyrone, a very fast release formulation for this active principle (more than 85% in 15 minutes) according to the present invention (ADF dipyrone+codeine 1000/30 mg/tablet) in 0.1 N HCl medium, pH 1.2, apparatus 2 (paddle) at 50 rpm rotation and 37 degrees, in 900 mL.

FIG. 2 presents the comparative dissolution profile of tablets containing 1000 mg of dipyrone of a very fast release formulation (more than 85% in 15 minutes), in HCl 0.1 N medium, pH 1.2, apparatus 2 (paddle), at 50 rpm rotation and 37 degrees, in 900 mL.—curve obtained with the reference medicine containing only this active principle, marketed under the brand name Novalgina®, by Sanofi Aventis group (1000 mg/cp. Batch No. 232011).

FIG. 3 presents the dissolution profile of tablets comprising 30 mg of codeine, a fast release formulation for this active principle (more than 85% in 30 minutes) according to the present invention (ADF dipyrone+codeine 1000/30 mg/tablet) in 0.1 N HCl medium, pH 1.2, apparatus 2 (paddle) at 50 rpm rotation and 37 degrees, in 900 mL.

FIG. 4 presents the comparative dissolution profile of tablets containing 30 mg of codeine of a fast release formulation (more than 85% in 30 minutes), in HCl 0.1 N medium, pH 1.2, apparatus 2 (paddle), at 50 rpm rotation and 37 degrees, in 900 mL.—curve obtained with the reference medicine containing only this active principle, marketed under the brand name Codeine, by Cristália group (30 mg/cp. Batch No. 12053389).

FIG. 5 presents the dissolution profile of tablets comprising 30 mg of codeine, a fast release formulation for this active principle (more than 85% in 30 minutes) according to the present invention (ADF dipyrone+codeine 500/30 mg/tablet), in 0.1 N HCl medium, pH 1.2 apparatus 2 (paddle), at 50 rpm rotation and 37 degrees, in 900 mL.

FIG. 6 presents the dissolution profile of tablets comprising 500 mg of dipyrone, a very fast release formulation for this active principle (more than 85% in 15 minutes) according to the present invention (ADF dipyrone+codeine 500/30 mg/tablet), in 0.1 N HCl medium, pH 1.2, apparatus 2 (paddle) at 50 rpm rotation and 37 degrees, in 900 mL.

FIG. 7 presents the comparative dissolution profile of tablets containing 500 mg of dipyrone of a very fast release formulation (more than 85% in 15 minutes), in HCl 0.1 N medium, pH 1.2, apparatus 2 (paddle), at 50 rpm rotation and 37 degrees, in 900 mL.—curve obtained with the reference medicine containing only this active principle, marketed under the brand name Novalgina®, by Sanofi Aventis group (500 mg/cp. Batch No. 233598).

FIG. 8 presents the stability profile of dipyrone in tablets containing 1000 mg of dipyrone+30 mg of codeine in a long-term study.

FIG. 9 presents the stability profile of codeine in tablets containing 1000 mg of dipyrone+30 mg of codeine in a long-term study.

FIG. 10 presents the stability profile of dipyrone in tablets containing 500 mg of dipyrone+30 mg of codeine in a long-term study.

FIG. 11 presents the stability profile of codeine in tablets containing 500 mg of dipyrone+30 mg of codeine in a long-term study.

FIG. 12 presents the stability profile of dipyrone in tablets containing 1000 mg of dipyrone+30 mg of codeine in an accelerated study.

FIG. 13 presents the stability profile of codeine in tablets containing 1000 mg of dipyrone+30 mg of codeine in an accelerated study.

FIG. 14 presents the stability profile of dipyrone in tablets containing 500 mg of dipyrone+30 mg of codeine in an accelerated study.

FIG. 15 presents the stability profile of codeine in tablets containing 500 mg of dipyrone+30 mg of codeine in an accelerated study.

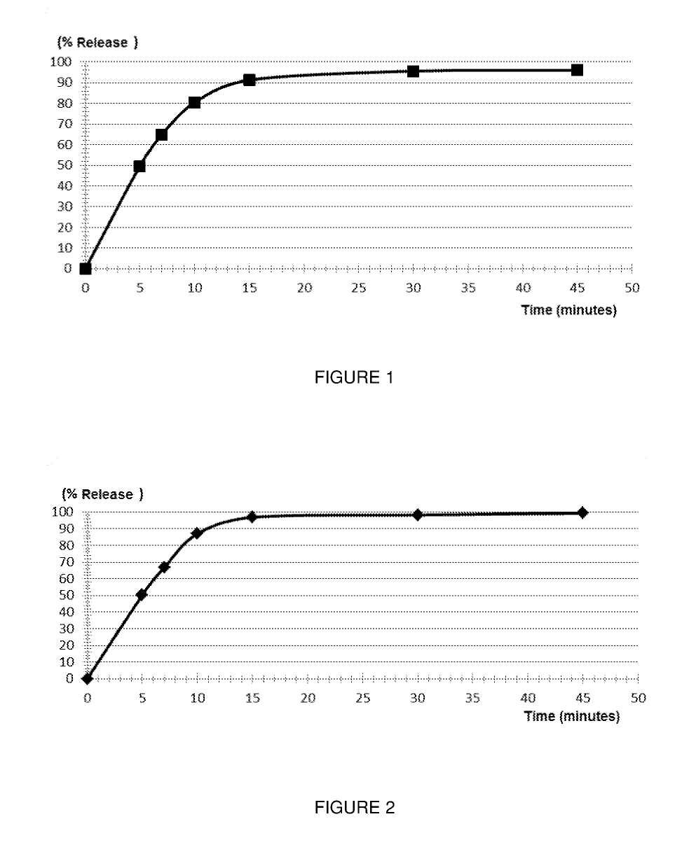

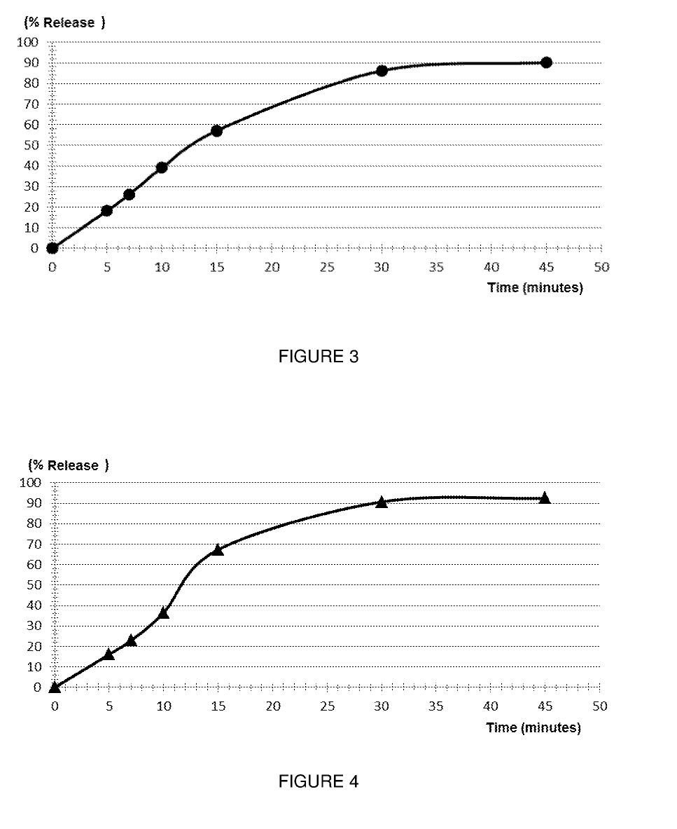

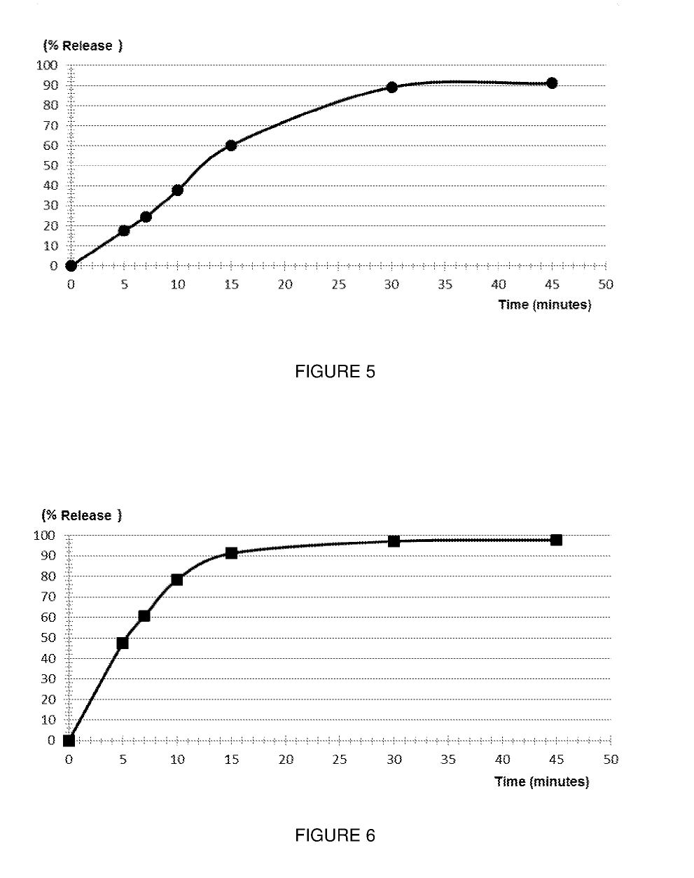

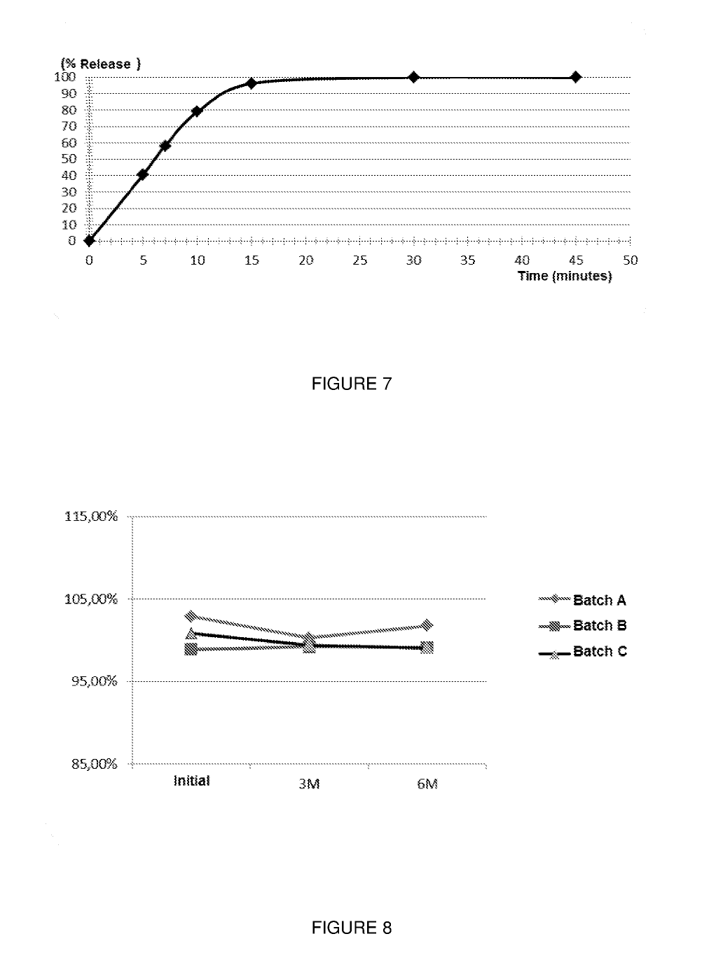

### DESCRIPTION OF THE INVENTION

Surprisingly, the pharmaceutical composition according to the present invention allows the association of two active principles with different profiles of pH stability: the non-narcotic anti-inflammatory analgesic dipyrone, stable at alkaline pH, and the opioid analgesic codeine, stable at acidic pH.

Then, the present invention combines the central action analgesic effects attributed to opioid analgesics with those predominantly peripheral, attributed to non-narcotic anti-inflammatory analgesics.

This object is achieved through the selection of ingredients and adequate pH range, allowing to carry, in the same pharmaceutical form, two active principles that require different pHs to preserve stability, so as to attain synergy of analgesic effects for pain suppression, particularly from moderate to intense, arising from trauma (sprains, dislocations, contusions, distensions, fractures), postoperative and post-tooth extraction conditions, neuralgia, lumbago, joint pains and similar conditions.

Thus, the present invention relates to stable oral pharmaceutical compositions characterized by comprising (a) dipyrone, its free acid or pharmacologically acceptable salts thereof, (b) codeine or pharmacologically acceptable salts thereof, (c) polyethylene glycol and (d) pharmaceutically acceptable excipients in small quantities, being that the compositions have a pH of about 6.5.

According to the present invention, the term “oral pharmaceutical compositions” means monolithic systems, such as single or bilayer tablets, simple or coated, capsules and multiparticulate systems, among others. Particularly, the present invention comprises coated tablets.

The preferably used dipyrone (a) is the dipyrone sodium monohydrate. On the other hand, codeine (b) is preferably used in the form of a codein phosphate hemihydrate.

Still in accordance with the present invention, polyethylene glycol or PEG (c) is a polymer derived from ethylene glycol, used as a binder, antihydrolytic and nonadherent. In a particular embodiment, polyethylene glycol has a molecular weight ranging between 1000 and 8000.

Still particularly, the present invention comprises a stable oral pharmaceutical composition comprising between 250 and 1,000 mg of dipyrone, between 7.5 and 30 mg of codeine, between 1.0 and 15% w/w of polyethylene glycol.

Moreover, since the pharmaceutical composition according to the present invention requires the use of significant amounts of active principles and considering the need to establish an oral composition easily ingested by the patient, the use of excipients in small quantities in the formulation is proposed.

Thus, once the invention proposes the use of expressive amounts of the non-narcotic anti-inflammatory analgesic (ratios between 250 and 1000 mg) compared to the opioid analgesic, the physical compatibility (compressibility, homogeneity) and chemical compatibility of the active principles, with the use of reduced amounts of excipients, is also a surprising aspect of the present invention.

The pharmaceutical composition, according to the present invention, may be prepared so that the active principles (a) and (b) can be comprised in the same or different granules.

In the first case, in a particular embodiment of the present invention, the active principles are contained in the same granule and can be produced by a wet granulation process, in which the active principle (b) is dissolved and stabilized in a binder solution, to be sprinkled on the active principle (a). Thus, the uniformity of the active ingredient in a smaller proportion and the obtainment of the oral composition in a size suitable for ingestion are guaranteed.

The following are examples of stabilizers for the binder solution: the antioxidants sodium metabisulfite and sodium bisulfite and the chelant sodium edetate or mixtures thereof, but they are not limited to these. The polyethylene glycols (PEG) are used as binders.

It was verified that said binder solution containing (b) has a pH near 4.0 which, in turn, promotes the stability of this active principle.

The use of polyethylene glycols is proposed, since it has been verified that the same, when are in solution, act not only as binders in the granulation process, but also effectively contribute to the increase of density of the granules, reducing the volume originally occupied by the active principles (which have low density) and thus enabling the obtainment of the composition with a size suitable for ingestion. Additionally, they act as antihydrolytic agents, equally contributing to the stability of the active principles and, since they are non-ionic surfactants, they increase drug solubility, optimizing its dissolution.

The particular use of PEG in solution, along with the active principle (b), demonstrated the occasional coating of this active principle after drying, providing certain degree of isolation of the same in relation to dipyrone. A nonadherent effect was also observed on the obtained granulate, thus avoiding adherence and issues arising from it during compression, often observed for the active principles under discussion. Therefore, the use of this excipient favors both physical and chemical compatibility of the formulation proposed.

Moreover, it has been verified through testing that the granulation of the active principle dipyrone with the binder solution proposed, despite of having acidic character to ensure greater stability of the active principle (b), does not promote any degradation of dipyrone. This occurs since this is an ephemeral process that, followed by drying of the granulate, inhibits the degradation of this drug by hydrolysis.

The final pH obtained for the formulation proposed in this particular embodiment of the invention is around 6.5, an intermediate value that ensures greater stability for both drugs (neither strongly acid to catalyze the hydrolysis of dipyrone, nor strongly alkaline to catalyze the degradation of codeine).

In the second case, in another particular embodiment of the invention, dipyrone (a) and codeine (b) can be comprised in granules separately produced. Thus, the specific aspects related to the stability of each active principle are also advantageously respected, since both are physically separated, enabling the best use of their features.

Particularly in this case, the granules containing the active principle dipyrone are wetly produced, through spraying with a binder solution comprised by stabilizers, such as the antioxidants sodium metabisulfite and sodium bisulfite, and the chelant sodium edetate, or mixtures thereof, among others; and polyethylene glycols. Similarly to what has been previously observed, the use of stabilizers favorably contributes to the stability of the drug under discussion, and the use of PEG promotes the increase of the granulate density in greater proportion in the product object of the invention.

The granulation and drying processes according to the present invention can be respectively carried out, for example, under high shear and in fluidized bed.

The granulate comprising the active principle codeine (b) can be produced, for example, with lactose, cellulose and starch, among other diluents, in small concentration. Stabilizers, such as the antioxidants sodium metabisulfite and sodium bisulfite can be included into the mixture, as well as the chelant sodium edetate or mixtures thereof, among others.

Povidones can be used as binders, but they are not limited to these. To make the granulation process easier and prevent adhesion of the active ingredient to the equipment, colloidal silica and talc, among other nonadherent agents, may be used.

To isolate an active ingredient from the other, it is proposed that the granulate containing the active ingredient codeine (b) should be coated with an inert polymer. In this case, a polymer having a hydrophilic coating and being water-permeable is used as an inert polymer, such as hydroxypropyl methyl cellulose, which can be an isolated polymer or be comprised in a mixture with polyethylene glycol; being dispersed in ethyl alcohol, water or an hydro alcoholic solution. Particularly, the hydrophilic coating and water-permeable polymer is preferably a mixture of hydroxypropyl methyl cellulose and polyethylene glycol dispersed in an hydro alcoholic solution.

The isolation of the active ingredient codeine (b) favors not only the chemical compatibility of the same with the dypirone granulate (a), since both molecules of the active principles present greater stability under different conditions of pH, and also promotes physical compatibility of the composition, since the active principles are hardly compressible.

Similarly to the first particular embodiment of the invention, in this second embodiment the final pH of the formulation is nearly 6.5, an intermediate value that ensures greater stability of the active principles.

In both embodiments proposed in the invention, disintegrants can be employed, such as crospovidone, starch, sodium starch glycolate, sodium croscarmellose, cellulose derivatives or mixtures thereof. Lubricants and glidants are also used, for example, hydrogenated vegetable oils, magnesium stearate, stearic acid, sodium stearyl fumarate, colloidal silicon dioxide, talc and/or mixtures thereof, but not being limited to these. Crospovidone and/or sodium croscarmellose are preferred disintegrants, which can be added in or out of the granule. On the other hand, a preferred lubricant is magnesium stearate or sodium stearyl fumarate, and/or mixtures of magnesium stearate with colloidal silicon dioxide or sodium stearyl fumarate with colloidal silicon dioxide.

The compression of the granulate(s) can be done in rotary compressors, and the shape and size of the tablets may vary.

The coat of the tablet aims to preserve stability of the active principles by protection provided by core isolation from the external environment. Preferably, the coating is hydrophilic, permeable to water and to the moist environment of the gastrointestinal tract.

The materials that form the coating are, e.g., mixtures of polyvinylpyrrolidone or polyvinylpyrrolidone-polyvinyl acetate copolymer with hydroxypropylmethylcellulose (HPMC) or polyvinyl alcohol. These materials that form coatings are dispersed in ethyl alcohol, water or hydro alcoholic mixtures to compose the application vehicle. Pigments, talc or wetting agents can be added to the polymer solution thus formed in water or alcohol.

Particularly, the coating is preferably made with the polyvinyl alcohol polymer dispersed in aqueous solution, with addition of opacifiers and plasticizers, such as polyethylene glycols, but without being limited to these.

The coating forming material may be sprinkled on the tablet as a dispersion, using perforated rotary coating equipments.

The present invention is further illustrated by the following examples, without limitation.

### Example 1

### Coated Tablet with a Single Granulate: Dipyrone 500 mg/Codeine 30 mg

TABLE 1
GRANULATE

| Ingredient | Amount (mg) |
|---|---|
| dipyrone sodium monohydrate | 500.00 |
| codein phosphate hemihydrate | 30.68 |
| sodium metabisulfite | 0.83 |
| polyethylene glycol | 16.68 |
| colloidal silicon dioxide | 2.93 |
| magnesium stearate | 5.56 |
| polyvinyl alcohol/PEG/talc | 17.79 |
| titanium dioxide | 4.45 |
| water | qsp |

Preparation Process

The dipyrone sodium monohydrate is moisturized and granulated in a high shear granulator, with aqueous solution of codeine phosphate hemihydrate, sodium metabisulfite and polyethylene glycol. The granules are dried in fluidized bed and standardized in a tapered granulator, through an appropriate mesh.

The obtained granulate is transferred to the mixer and colloidal silicon dioxide is added, previously passed through an adequate mesh and homogenized for an adequate period of time. Then, magnesium stearate previously sieved through an adequate mesh is added to the mixer and homogenized.

After compression in a rotary compressor, the coating is carried out through a circular equipment with perforated bucket.

The coating used is a mixture comprised by polyvinyl alcohol, titanium dioxide, PEG and talc.

As it is verified in the figures, the composition according to the present invention provides adequate dissolution in comparison to the individual profiles of commercial medicines Novalgina® and Codeine®, which use the active principles separately.

### Example 2

### Coated Tablet with a Single Granulate: Dipyrone 1000 mg/Codeine 30 mg

TABLE 2
GRANULATE

| Ingredient | Amount (mg) |
|---|---|
| dipyrone sodium monohydrate | 1,000.00 |
| codein phosphate hemihydrate | 30.68 |
| sodium metabisulfite | 1.62 |
| polyethylene glycol | 32.60 |
| colloidal silicon dioxide | 4.98 |
| magnesium stearate | 10.80 |
| polyvinyl alcohol/PEG/talc | 34.56 |
| titanium dioxide | 8.64 |
| water | qsp |

Preparation Process

The dipyrone sodium monohydrate is moisturized and granulated in a high shear granulator, with aqueous solution of codeine phosphate hemihydrate, sodium metabisulfite and polyethylene glycol. The granules are dried in fluidized bed and standardized in a tapered granulator, through an appropriate mesh.

The obtained granulate is transferred to the mixer and colloidal silicon dioxide is added, previously passed through an adequate mesh and homogenized for an adequate period of time. Then, magnesium stearate previously sieved through an adequate mesh is added to the mixer and homogenized.

After compression in a rotary compressor, the coating is carried out through a circular equipment with perforated bucket.

The coating used is a mixture comprised by polyvinyl alcohol, titanium dioxide, PEG and talc.

The comparison between the curves of the tablet dissolution profiles, according to the present invention and the state of the art product shows a similar in vitro performance in comparison with the individual profiles of medicines Novalgina® and Codeine®, which comprise the active principles individually.

### Example 3

### Coated Tablet with a Single Granulate Using Disintegrant: Dipyrone 1000 mg/Codeine 30 mg

TABLE 3
GRANULATE

| Ingredient | Amount (mg) |
|---|---|
| dipyrone sodium monohydrate | 1,000.00 |
| codein phosphate hemihydrate | 30.68 |
| sodium metabisulfite | 1.71 |
| polyethylene glycol | 34.45 |
| sodium croscarmellose | 57.00 |
| colloidal silicon dioxide | 5.44 |
| magnesium stearate | 11.40 |
| polyvinyl alcohol/PEG/talc | 36.48 |
| titanium dioxide | 9.12 |
| water | qsp |

Preparation Process

The mixture comprising dipyrone sodium monohydrate and part of the sodium croscarmellose is moisturized and granulated in a high shear granulator, with aqueous solution of codeine phosphate hemihydrate, sodium metabisulfite and polyethylene glycol. The granules are dried in fluidized bed and standardized in a tapered granulator, through an appropriate mesh.

The obtained granulate is transferred to the mixer and the remainder of sodium croscarmellose and colloidal silicon dioxide is added, previously passed through an adequate mesh and homogenized for an adequate period of time. Then, magnesium stearate previously sieved through an adequate mesh is added to the mixer and homogenized.

After compression in a rotary compressor, the coating is carried out through a circular equipment with perforated bucket.

The coating used is a mixture comprised by polyvinyl alcohol, titanium dioxide, PEG and talc.

The comparison between the curves of the tablet dissolution profiles, according to the present invention and the state of the art product shows a similar in vitro performance in comparison with the individual profiles of medicines Novalgina® and Codeine®, which comprise the active principles individually.

### Example 4

### Tablet Coated in Separate Granules: Dipyrone 1000 mg/Codeine 30 mg

TABLE 4
GRANULATE 1

| Ingredient | Amount (mg) |
|---|---|
| dipyrone sodium monohydrate | 1,000.00 |
| sodium metabisulfite | 1.70 |
| polyethylene glycol | 35.00 |
| water | qsp |

Manufacturing Process

The dipyrone sodium monohydrate is moisturized and granulated in a high shear granulator, with aqueous solution of sodium metabisulfite and polyethylene glycol. The granules are dried in fluidized bed and standardized in a tapered granulator, through an appropriate mesh.

TABLE 5
GRANULATE 2

| Ingredient | Amount (mg) |
|---|---|
| codein phosphate hemihydrate | 30.68 |
| cellulose | 25.00 |
| sodium metabisulfite | 0.10 |
| povidone | 4.50 |
| hypromellose/PEG | 3.50 |
| talc | 2.50 |
| colloidal silicon dioxide | 5.25 |
| magnesium stearate | 11.17 |
| polyvinyl alcohol/PEG/talc | 35.87 |
| titanium dioxide | 8.97 |
| ethyl alcohol | qsp |
| water | qsp |

Manufacturing Process

The mixture of codeine phosphate, cellulose and talc is firstly moisturized and granulated with aqueous solution of sodium metabisulfite and povidone. The granules are dried in fluidized bed and then coated with a hydro alcoholic solution of the mixture comprising hypromellose and PEG. Subsequently, they are standardized in a tapered granulator, through an adequate mesh.

The two granules obtained are transferred to the mixer. Colloidal silicon dioxide passed through an adequate mesh is added, homogenizing for an adequate period of time. Then, magnesium stearate previously sieved through an adequate mesh is added to the mixer and homogenized. After compression in a rotary compressor, the coating is carried out through a circular equipment with perforated bucket.

The coating used is a mixture comprised by polyvinyl alcohol, titanium dioxide, PEG and talc.

Similarly to the other proposals, the comparison between the curves of the tablet dissolution profiles, according to the present invention, and the state of the art product shows a similar in vitro performance in view of its comparison with the individual profiles of medicines Novalgina® and Codeine®, which comprise the active principles individually.

### Example 5

### Tablet Coated in Separate Granules: Dipyrone 500 mg/Codeine 30 mg

TABLE 6
GRANULATE 1

| Ingredient | Amount (mg) |
|---|---|
| dipyrone sodium monohydrate | 500.00 |
| sodium metabisulfite | 0.85 |
| polyethylene glycol | 17.50 |
| water | qsp |

Manufacturing Process

The dipyrone sodium monohydrate is moisturized and granulated in a high shear granulator, with aqueous solution of sodium metabisulfite and polyethylene glycol. The granules are dried in fluidized bed and standardized in a tapered granulator, through an appropriate mesh.

TABLE 7
GRANULATE 2

| Ingredient | Amount (mg) |
|---|---|
| codein phosphate hemihydrate | 30.68 |
| cellulose | 25.00 |
| sodium metabisulfite | 0.10 |
| povidone | 4.50 |
| hypromellose/PEG | 3.50 |
| talc | 2.50 |
| colloidal silicon dioxide | 2.75 |
| magnesium stearate | 5.86 |
| polyvinyl alcohol/PEG/talc | 19.04 |
| titanium dioxide | 4.76 |
| ethyl alcohol | qsp |
| water | qsp |

Manufacturing Process

The mixture of codeine phosphate, cellulose and talc is firstly moisturized and granulated with aqueous solution of sodium metabisulfite and povidone. The granules are dried in fluidized bed and then coated with a hydro alcoholic solution of the mixture comprising hypromellose and PEG. Subsequently, they are standardized in a tapered granulator, through an adequate mesh.

The two granules obtained are transferred to the mixer. Colloidal silicon dioxide passed through an adequate mesh is added, homogenizing for an adequate period of time. Then, magnesium stearate previously sieved through an adequate mesh is added to the mixer and homogenized.

After compression in a rotary compressor, the coating is carried out through a circular equipment with perforated bucket.

The coating used is a mixture comprised by polyvinyl alcohol, titanium dioxide, PEG and talc.

Similarly to the other proposals, the comparison between the curves of the tablet dissolution profiles, according to the present invention, and the state of the art product shows a similar in vitro performance in view of its comparison with the individual profiles of medicines Novalgina® and Codeine®, which comprise the active principles individually.

### Example 6

### Tablet Coated in Separate Granules with Disintegrant: Dipyrone 1000 mg/Codeine 30 mg

TABLE 8
GRANULATE 1

| Ingredient | Amount (mg) |
|---|---|
| dipyrone sodium monohydrate | 1,000.00 |
| sodium metabisulfite | 1.75 |
| polyethylene glycol | 35.20 |
| crospovidone | 59.00 |
| colloidal silicon dioxide | 5.37 |
| water | qsp |

Manufacturing Process

The dipyrone sodium monohydrate is moisturized and granulated in a high shear granulator, with aqueous solution of sodium metabisulfite and polyethylene glycol. The granules are dried in fluidized bed, standardized in a tapered granulator, through an appropriate mesh, and transferred to the mixer. Crospovidone and colloidal silicon dioxide are added to the mixer, previously passed through an adequate mesh and homogenized for an adequate period of time.

TABLE 9
GRANULATE 2

| Ingredient | Amount (mg) |
|---|---|
| codein phosphate hemihydrate | 30.68 |
| cellulose | 25.09 |
| sodium metabisulfite | 0.11 |
| povidone | 10.00 |
| hypromellose/PEG | 7.00 |
| talc | 2.50 |
| magnesium stearate | 11.80 |
| polyvinyl alcohol/PEG/talc | 38.03 |
| titanium dioxide | 9.51 |
| ethyl alcohol | qsp |
| water | qsp |

Manufacturing Process

The mixture of codeine phosphate, cellulose and talc is firstly moisturized and granulated with aqueous solution of sodium metabisulfite and povidone. The granules are dried in fluidized bed and then coated with a hydro alcoholic solution of the mixture comprising hypromellose and PEG. Subsequently, they are standardized in a tapered granulator, through an adequate mesh.

The two granules obtained are transferred to the mixer. Magnesium stearate previously sieved through an adequate mesh is added and homogenized.

After compression in a rotary compressor, the coating is carried out through a circular equipment with perforated bucket.

The coating used is a mixture comprised by polyvinyl alcohol, titanium dioxide, PEG and talc.

Similarly to the other proposals, the comparison between the curves of the tablet dissolution profiles, according to the present invention, and the state of the art product shows a similar in vitro performance in view of its comparison with the individual profiles of medicines Novalgina® and Codeine®, which comprise the active principles individually.

### Example 7

### Stability Study of the Pharmaceutical Compositions

Stability study under the following conditions: 40° C./75% RH (accelerated stability) and 30° C./75% RH (long term stability). During this period, all indicative tests of stability and all possible impurities of the associated product are evaluated, in order to ensure a stable and safe composition for human use.

The tests demonstrated that the stabilization of the association under discussion, which deals with active components with distinct chemical characteristics, is possible through the use of constituents that, when associated, allow a pH of the matrix nearly neutral, thus ensuring great stability to the final pharmaceutical composition.

FIGS. 8 to 15 show the results obtained in the accelerated stability and of long duration studies for pilot lots of tablets containing 1000 mg of dipyrone +30 mg of codeine, and of tablets containing 500 mg of dipyrone +30 mg of codeine, packed in aluminum blisters (ALU-ALU).

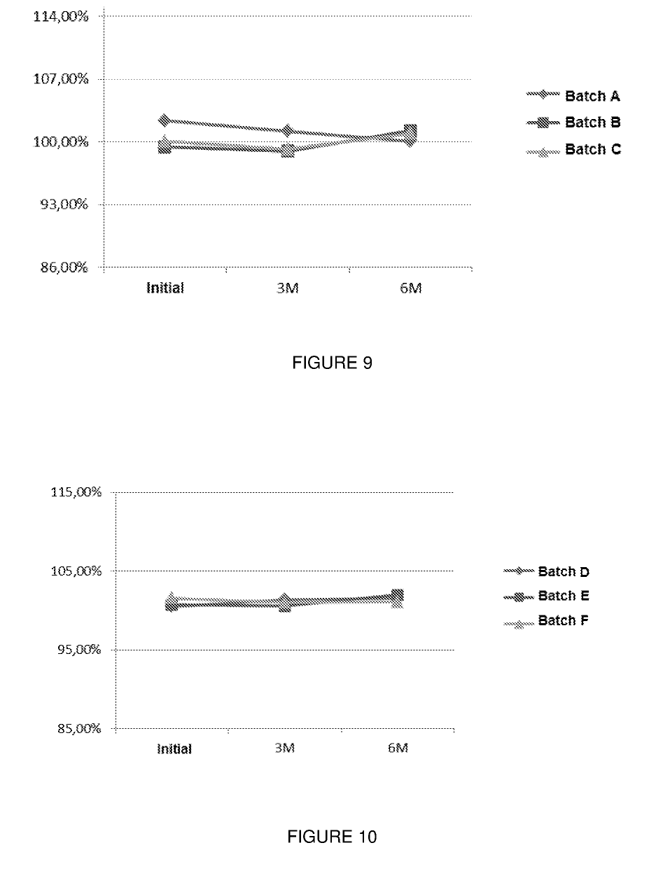

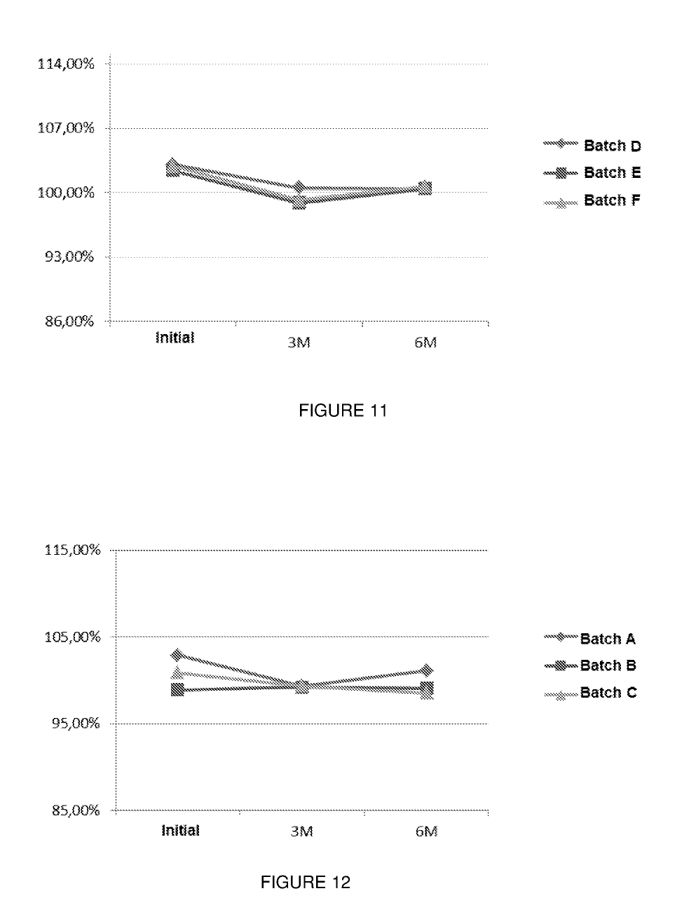

Taking into account the stability profile presented by both dypirone (FIG. 8) and codeine (FIG. 9) in tablets having 1000 mg of dipyrone+30 mg of codeine, it is verified that there was not significant variation in the content of batches after 3 and 6 months of testing, in the long-term study. The same result was found for tablets containing 500 mg of dipyrone+30 mg of codeine, as can be observed in the stability profile presented by dypirone (FIG. 10) and codeine (FIG. 11).

TABLE 10
Results of Compounds/Related Substances
1000 mg dipyrone + 30 mg codeine

| | | Accelerated | | | Long Term | | |
|---|---|---|---|---|---|---|---|
| Test | Specification | Batch A | Batch B | Batch C | Batch A | Batch B | Batch C |
| Individual | The area of any secondary | Not | Not | Not | Not | Not | Not |
| Impurities | peak should be less than | detected | detected | detected | detected | detected | detected |
| | 0.1% in relation to the total | | | | | | |
| | area of the chromatogram | | | | | | |
| Total | The sum of all individual | Not | Not | Not | Not | Not | Not |
| Impurities | impurities should be less | detected | detected | detected | detected | detected | detected |
| | than 1.0% | | | | | | |
| Morphine | No blue color is produced | In | In | In | In | In | In |
| Limit | immediately | conformity | conformity | conformity | conformity | conformity | conformity |

500 mg dipyrone + 30 mg codeine

| | | Accelerated | | | Long Term | | |
|---|---|---|---|---|---|---|---|
| Test | Specification | Batch D | Batch E | Batch F | Batch D | Batch E | Batch F |
| Individual | The area of any secondary | Not | Not | Not | Not | Not | Not |
| Impurities | peak should be less than | detected | detected | detected | detected | detected | detected |
| | 0.1% in relation to the total | | | | | | |
| | area of the chromatogram | | | | | | |
| Total | The sum of all individual | Not | Not | Not | Not | Not | Not |
| Impurities | impurities should be less | detected | detected | detected | detected | detected | detected |
| | than 1.0% | | | | | | |
| Morphine | No blue color is produced | In | In | In | In | In | In |
| Limit | immediately | conformity | conformity | conformity | conformity | conformity | conformity |

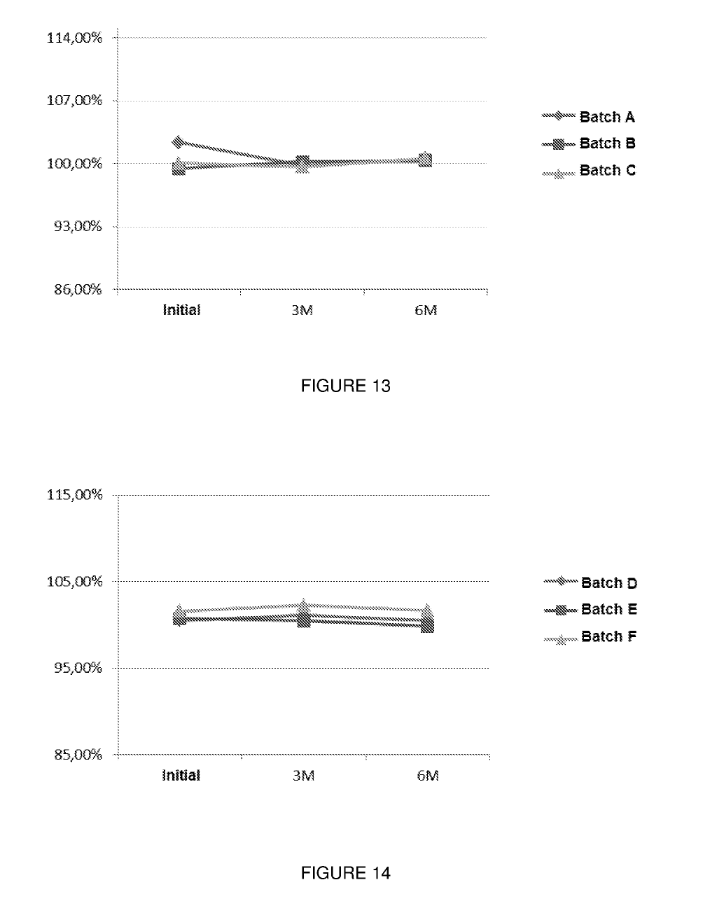

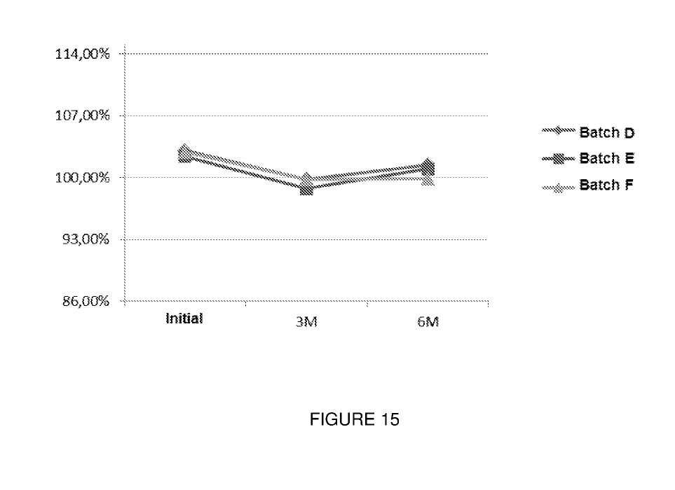

An accelerated study was carried out with the same lots of the previous study. The profile of dipyrone (FIG. 12) in tablets having 1000 mg of dipyrone +30 mg of codeine demonstrates the stability of the content of the batches after 3 and 6 months of test, as well as the profile of codeine (FIG. 13), which also remained stable. In tablets with 500 mg of dipyrone+30 mg of codeine, the levels of dipyrone (FIG. 14) and codeine (FIG. 15) also remained stable.

The results demonstrate excellent stability of the composition according to the present invention, which is guaranteed by the pH of the matrix nearly neutral. No significant variations were detected in the test of contents and impurities/related compounds were not found either during the evaluated period, as it is observed in table 7.

Compared to other performance parameters evaluated during the product stability (for example, dissolution), it should be pointed out that the same remained fulfilling the specification, thus guarantying a favorable stability to the product. It should be noted that similar results were obtained with products packed in ACLAR-aluminum blisters, demonstrating that the packaging material is not the limiting factor in the stability of the proposed product.

In addition, it can be affirmed, based on the results found, that the stability of the composition object of the invention is comparable to drugs that contain the active principles individually, such as Novalgina® and Codein®. Such comparability is possible due to the fact that such medicines are commercially available and that, for this reason, it is assumed that they meet all the criteria of quality and specifications established by pharmacopoeias and have been assayed by regulatory agencies in the context of being safe and of having acceptable quality to human use. Therefore, it is reaffirmed that despite all the difficulties to set forth the composition object of the invention, a product with satisfactory stability comparable to medicines Novalgina® and Codein® was obtained, but with the advantage of being a fixed-dose association which provides many benefits to human use, as has been already explained.

### Example 6

### Clinical Study to Evaluate Pharmacokinetics of the Compositions in Healthy Subjects

Test of relative bioavailability in order to detect possible pharmacokinetic interactions when associating active principles, providing information analyzed in conjunction with the clinical study to set forth the rationale of the association. Its purpose is the evaluation of the pharmacokinetic profile of each active ingredient, as well as the pharmacokinetic interaction of dipyrone sodium monohydrate with codeine phosphate. For this purpose, the composition according to the present invention with commercially available products are comparatively evaluated: dipyrone sodium monohydrate (Novalgina®), a simple 1000 mg tablet available from Sanofi-Aventis Farmacêutica Ltda; codeine phosphate (Codein), a simple 30 mg tablet available from Cristália Produtos Químicos Farmacêuticos Ltda.

The comparative bioavailability of the formulations after oral administration is evaluated based on statistical comparisons of relevant pharmaco-kinetic parameters obtained from data of concentrations to the drugs, from collected blood samples. The concentrations are measured through a previously standardized and validated analytical method.

Study Design

The formulations of simple coated tablets comprising 1000 mg of dipyrone sodium monohydrate and/or or 30 mg of codeine phosphate are orally administered in a single dose to male and female volunteers, which are healthy adults under fasting conditions. The balancing of volunteers based on sex is previously considered; therefore, the first 24 volunteers are of the same sex, and the other 24 of the other.

The formulations are orally administered in a single dose, followed by blood samplings comprising 3 to 5 half-life times of drug elimination. The interval between the doses has a minimum interval of 7 half-lives between them. Considering that the half-life is 2.6 to 3.5 hours to dipyrone and from 2.5 to 3 hours to codeine, there is a minimum interval of 7 days between the administrations. The study design is of 3 periods and 6 sequences, however, for statistical analysis, this design was dismembered into three other designs of 2 periods and 2 sequences.

Primary Outcome

The study has, as primary outcome, the descriptive and comparative analysis of the pharmaco-kinetic parameters obtained from plasma concentrations of dipyrone sodium monohydrate (through its clinically active metabolites: 4-MAA (4-N-methylaminoantipyrine) and 4-AA (4-aminoantipyrine)) and codeine phosphate, administered separately or in association.

Secondary Outcome

Evaluation of safety and tolerability through the report of adverse events.

Study Methodology

The study is designed to allow the obtainment of the pharmaco-kinetic parameters ASCO-t and Cmax for statistical comparison. Such parameters are obtained from the determination of plasma concentration of the drug after oral administration of the medicine, by applying the non-compartmental model for this type of study. The primary purpose of clinical stage is the collection of blood samples from volunteers after oral administration of the medicine, and sending of these samples to the analytical stage, which quantifies the drug concentration in plasma related to each collection time.

Analytical Step

The plasma concentrations of dipyrone sodium monohydrate (through its clinically active metabolites: 4-MAA (4-N-methylaminoantipyrine) and 4-AA (4-aminoantipyrine), and of codeine phosphate, are quantified by a specific analytical method previously validated, based on liquid chromatography coupled to mass spectrometry (LC-MS/MS) or another detector, at the discretion of the analytical laboratory.

Statistical Treatment

Experimental Design. The design used for this study is a 3×6 open and randomized crossover design for three treatments (test medicine=T, reference medicine 1=R1 and reference medicine 2=R2), three periods (1, 2 and 3) and, consequently, six sequences (T, R2, R1; R1, T, R2; R2, R1, T; R1, R2, T; R2, T, R1; T, R1, R2) of medicines. In this design, each block (individual) receives three treatments in distinct periods, and the medicine sequences are randomly assigned to individuals in a balanced manner, in order to minimize the effects of sequence and period. The balance between sexes also occurs due to the participation of male and female research subjects in this study. The time interval, to ensure that the residual effect of the administered formulation in period 1 was eliminated until period 2 and 3 (washout) is of at least 7 days.

Sample Size

The number of volunteers selected for this study (sample size) is estimated in anticipation of possible drop-outs and exclusions, thus guarantying the reliability of statistical results. To preserve the sample size (N) of the study, the gathered volunteers that could not be admitted in the first period could be replaced by reserve volunteers.

Analysis of Pharmacokinetic Parameters

The following pharmaco-kinetic parameters were determined after the quantification of plasma concentrations in the analytical step of the study:

Cmax Maximum concentration detected in plasma after each treatment (T, R1 and R2).

ASCO-t Area under the concentration curve versus time, from zero to the last concentration experimentally determined.

Asc0-¥ Area under the concentration curve versus time, from zero to infinity.

Tmax Time corresponding to the maximum plasma concentration.

Kel Elimination constant of terminal phase.

T1/2 Half-life elimination time of the drug.

Pharmacokinetic parameters Tmax, Cmax and ASC were taken into account in the descriptive and comparative analysis.

The scale (original or logarithmic) in which the data are statistically evaluated is determined after the verification of the assumptions required for the application of statistical methods through a descriptive analysis and residue analysis of the crossover model. An analysis of variance (ANOVA) is carried out to evaluate the effects of sequence, treatment and period on the pharmacokinetic parameters Cmax, ASCO-t and ASCO-8, as well as to check if there was difference between them. Pharmacokinetics can be described through the ratio between the arithmetic averages (original scale) or through the ratio of geometric averages (logarithmic scale) of the test and reference products, for the pharmaco-kinetic parameters Cmax and ASCO-t. The pharmacokinetic description between T, R1 and R2 is made by a 90% confidence interval (90% CI) for these ratios. The statistical analysis between the medicines T, R1 and R2 is based on three 2×2 crossover analyses derived from the randomization dismemberment.

Analysis of Safeness and Tolerability

The descriptive analysis of adverse events that occurred during the study was performed, identified by:

Total of Adverse Events

Total Number of Volunteers who had Adverse Events

Amount and Percentage of Adverse Events per Period

Amount and Percentage of Adverse Events per Period and in Relation to the Drug

Amount and Percentage of Adverse Events by Type of Medicine

Amount and Percentage of Adverse Events by Type of Medicine and in Relation to the Drug

It shall be understood that the embodiments described above are merely illustrative and any modification to them may occur for a person skilled in the art. Therefore, the present invention should not be considered as being limited to the embodiments described in this document.

The person skilled in the art can readily evaluate, by means of the teachings contained in the text and in the presented examples, advantages of the invention, and propose modifications and equivalent alternatives to the embodiments, without departing from the scope of the invention, as defined in the attached claims.

1. STABLE ORAL PHARMACEUTICAL COMPOSITION, CHARACTERIZED by the fact that it comprises (a) dipyrone, its free acid or pharmacologically acceptable salts thereof, (b) codeine or pharmacologically acceptable salts thereof, (c) polyethylene glycol and (d) pharmaceutic excipients.

2. STABLE ORAL PHARMACEUTICAL COMPOSITION, according to claim 1, CHARACTERIZED by the fact that the composition has pH of about 6.5.

3. STABLE ORAL PHARMACEUTICAL COMPOSITION, according to claim 1, CHARACTERIZED by the fact that dipyrone (a) is dipyrone sodium monohydrate.

4. STABLE ORAL PHARMACEUTICAL COMPOSITION, according to claim 1, CHARACTERIZED by the fact that codeine (b) is codein phosphate hemihydrate.

5. STABLE ORAL PHARMACEUTICAL COMPOSITION, according to claim 1, CHARACTERIZED by the fact that polyethylene glycol (c) is selected from one or more polymers with molecular weight ranging between 1000 and 8000.

6. STABLE ORAL PHARMACEUTICAL COMPOSITION, according to claim 1, CHARACTERIZED by the fact that it contains from about 250 to about 1,000 mg of dipyrone (a).

7. STABLE ORAL PHARMACEUTICAL COMPOSITION, according to claim 1, CHARACTERIZED by the fact that it contains from about 7.5 to about 30 mg of codeine (b).

8. STABLE ORAL PHARMACEUTICAL COMPOSITION, according to claim 1, CHARACTERIZED by the fact that it contains from about 1.0% to about 15.0% w/w of polyethylene glycol.

9. STABLE ORAL PHARMACEUTICAL COMPOSITION, according to claim 1, CHARACTERIZED by the fact that the pharmaceutically acceptable excipients (d) comprise stabilizers, lubricants, diluents, binders, disintegrants, inert polymer and/or coating forming materials.

10. STABLE ORAL PHARMACEUTICAL COMPOSITION, according to claim 9, CHARACTERIZED by the fact that the stabilizers are selected from one or more of: sodium metabisulfite, sodium bisulfite, sodium edetate or mixtures thereof.

11. STABLE ORAL PHARMACEUTICAL COMPOSITION, according to claim 9, CHARACTERIZED by the fact that the lubricants are selected from one or more of: hydrogenated vegetable oils, magnesium stearate, stearic acid, sodium stearyl fumarate, colloidal silicon dioxide, talc or mixtures thereof.

12. STABLE ORAL PHARMACEUTICAL COMPOSITION, according to claim 9, CHARACTERIZED by the fact that the diluents are selected from one or more of: lactose, cellulose, starch or mixtures thereof.

13. STABLE ORAL PHARMACEUTICAL COMPOSITION, according to claim 9, CHARACTERIZED by the fact that the binders are selected from one or more of: povidone, copovidone, PEG, starch, hydroxypropylmethylcellulose and other cellulose derivatives, or mixtures thereof.

14. STABLE ORAL PHARMACEUTICAL COMPOSITION, according to claim 9, CHARACTERIZED by the fact that the disintegrants are selected from one or more of: crospovidone, starch, sodium starch glycolate, sodium croscarmellose, cellulose derivatives or mixtures thereof.

15. STABLE ORAL PHARMACEUTICAL COMPOSITION, according to claim 9, CHARACTERIZED by the fact that the inert polymer and/or coating forming materials are selected from one or more of: hydroxypropylmethylcellulose, hydroxypropylmethylcellulose/PEG, polyvinylpyrrolidone, polyvinylpyrrolidone copolymer, polyvinyl acetate with hydroxypropylmethylcellulose, polyvinyl alcohol or mixtures thereof.

16. STABLE ORAL PHARMACEUTICAL COMPOSITION, according to claim 15, CHARACTERIZED by the fact that the inert polymer and/or coating forming materials are dispersed in ethyl alcohol, water or hydro alcoholic mixtures.
